(19) United States
(12) Patent Application Publication
(10) Pub. No.: US 2025/0170174 A1
(43) Pub. Date: May 29, 2025

(54) METHODS OF TREATING LEUKEMIA

(71) Applicant: Mor Research Applications Ltd., Ramat Gan (IL)

(72) Inventor: Uri Rozovski, Ramat Gan (IL)

(21) Appl. No.: 18/523,442

(22) Filed: Nov. 29, 2023

(51) Int. Cl. A61K 35/17; A61K 45/06; A61P 35/02

(52) U.S. Cl. CPC A61K 35/17; A61K 45/06; A61P 35/02

(57) ABSTRACT

Methods for treating leukemia are disclosed, comprising administering to a subject in need thereof therapeutically effective amounts of natural killer (NK)-derived exosomes. One non-limiting practical utilization of the NK-derived exosomes is induction therapy of acute myeloid leukemia (AML), to thereby immediately and effectively induce remission of the disease in newly diagnosed AML patients.

### FIELD OF THE INVENTION

The present disclosure relates to the therapeutical application of exosomes in the treatment of cancer, specifically, to natural killer cell-derived exosomes as a cell-free therapy for leukemia.

### BACKGROUND

Natural killer (NK) cells are innate lymphoid cells that play a central role in tumor immune surveillance. Unlike T-cells and B-cells, NK-cells do not require prior antigen sensitization, therefore, they possess the ability to lyse target cells in an antigen-independent non-specific manner. Nevertheless, the wide use of NK cells-based therapy is limited for several reasons: (i) NK cells-based products are potentially immunogenic; (ii) safety-related procedures such as irradiation of the cells may compromise their efficacy; and (iii) leukemia cells immunosuppress NK cells thus severely mitigating their cytotoxicity.

Recently, exosomes have emerged as a powerful, natural therapeutic tool. Exosomes are nanosized membranous particles secreted by all cells, that carry a cargo of proteins and nucleic acids and participate in a wide range of biological processes. Being natural transporters, exosomes interact with cellular membranes, and deliver their payload to target cells. Compared with cell-based therapies, exosomes are considered safer since they lack proliferation capacity, nulling the need to irradiate them. Moreover, exosomes are potentially less immunogenic and less cytotoxic and can thus be delivered at higher doses and increased frequencies. Exosomes offer significant advantages as cancer therapeutics, owing to their size, surface expression profiles, targeting capability, low immunogenicity, low cytotoxicity, long-term safety and prolonged circulatory half-life.

### SUMMARY

The present disclosure circumvents problems associated with harnessing natural killer (NK) cells for defeating cancerous processes by providing means and methods for utilizing NK-derived exosomes (NKexo) as the therapeutic agents.

Treatment regimens for acute myeloid leukemia (AML) fail in almost 50% of patients, and the survival rate has been only marginally raised during the past 4 decades. Currently, no effective immunologic induction therapy for AML is available or known. The hemato-oncology community envisioned that the introduction of chimeric antigen receptor (CAR) treatment about a decade ago would be a “game changer”. However, the time needed to produce effective CAR/NK is detrimental for AML patients, and even if this time gap could be overcome, the use of autologous NK may fail to recognize the AML cells as non-self and irradicate it.

Embodiments described herein relate to the successful harnessing of exosomes derived from NK cells in selectively and effectively treating leukemia in animal models. Specifically, it is shown herein that exosomes which were kept frozen and then thawed and immediately administered to the diseased animals, enabled effective treatment of AML.

These findings provide, for the first time, pre-clinical evidence needed to test the NK-derived exosome approach in clinical studies and ultimately develop an acellular method for treating leukemia.

In one aspect, the present disclosure relates to a method for treating leukemia, comprising administering to a subject in need thereof therapeutically effective amount of NK-derived exosomes. The NKexo may be administered at a dose which accounts for 1 to 4 μg exosomal protein per treatment course.

The NK-derived exosomes may be autologous and/or allogenic, for example NK cell-line derived exosomes or exosomes obtained from a living donor. In some embodiments, NKexo are obtained from NK cell-line.

The NKexo administered to a subject in need thereof may be freshly prepared/harvested or preserved exosomes. In some embodiments, preserved NKexo are frozen exosomes.

A disclosed method may be applied for the treatment of various types of leukemia, including, but not limited to acute lymphocytic leukemia (ALL), chronic lymphocytic leukemia (CLL), chronic myeloid leukemia (CML), chronic myelomonocytic leukemia (CMML) and acute myeloid leukemia (AML).

In another aspect, the present disclosure relates to a method for induction therapy in a subject afflicted with AML, comprising administering therapeutically effective amount of NK-derived exosomes to the subject to thereby induce remission.

The NKexo protein administered per treatment course may range from about 1 to about 3 μg, wherein one course of treatment is 7 days.

In some embodiments, the NKexo are non-autologous, for example, exosome derived from NK cell-lines.

A disclosed method may optionally be combined with standard of care anti-cancer therapy such as but not limited to chemotherapy, immunotherapy, irradiation and bone marrow transplantation.

### BRIEF DESCRIPTION OF THE DRAWINGS

The patent or application file contains at least one drawing executed in color. Copies of this patent or patent application publication with color drawings will be provided by the Office upon request and payment of the necessary fee.

Some embodiments are herein described, by way of example only, with reference to the accompanying drawings. With specific reference now to the drawings in detail, it is stressed that the particulars shown are by way of example and for purposes of illustrative discussion of embodiments. In this regard, the description taken with the drawings makes apparent to those skilled in the art how embodiments may be practiced.

In the drawings:

FIGS. 1A-1D show isolation and characterization of extracellular vesicles (EVs) derived from NK92MI cells. 1A: a representative output of nanoparticle tracking analysis (NTA) of NK92MI-derived EVs showing concentration and size distribution. 1B: a transmission electron microscopes (TEM) analysis image of NK92MI-derived EVs (scale bar, 100 nm). 1C: flow cytometry histograms of isolated vesicles stained with the exosomal markers CD63 and CD81 or with the NK marker CD56. 1D: an image of Western blot of NK92MI cell- and NK92MI EV-lysates (NKexo);

FIGS. 2A-2D show the natural killer cells-derived exosomes (NKexo)-induced selective apoptosis in leukemia cells. 2A: representative flow cytometry dot plots of chronic myeloid leukemia (K562), acute lymphocytic leukemia (Jurkat, UoC) and acute myeloid leukemia (KG-1a, HL-60) cell-lines exposed to 20 μg of NKexo. The apoptosis rate was assessed by annexin-V/propidium iodide (PI) staining. Cells in the lower right quadrant indicate annexin V-positive, early apoptotic cells. The cells in the upper right quadrant indicate annexin-positive/PI-positive, late apoptotic cells or necrosis. Cells in the lower left quadrant indicate annexin-negative/PI-negative, viable cells. 2B: a bar graph of the percentages of apoptotic cells following 4 h of 20 μg NKexo treatment with or without 20 μM z-VAD. 2C: representative Western blot of caspase-3, cleaved caspase-3 and tubulin in HL-60 cells treated or untreated with NKexo (10 μg or 20 μg) in the presence or absence of z-VAD (25 μM). 2D: a graph showing the rate of HL-60 cells apoptosis following exposure to 5 or 20 μg NKexo for 0, 2, 4, 10, and 24 h. The rate of cells in apoptosis was assessed by annexin-V/PI staining. Values represent a mean of three experiments ±SEM. *p≤0.05; **p≤0.005;

FIGS. 3A-3E show cytolytic activity of NKexo on various leukemia cell-lines. 3A-3B: representative fluorescent images of calcein-acetoxymethyl ester (AM) (red (3A) or green (3B)) stained chronic myeloid leukemia (K562), acute lymphocytic leukemia (Jurkat, UoC) and acute myeloid leukemia (KG-1a, HL-60) cell-lines and immortalized B-cells, exposed to 5 or 20 μg of NKexo in the presence or 25 μM of the pan-caspase inhibitor z-VAD (3B) or absence thereof (3A), for the indicated times (10× magnification). 3C: a bar graph showing the percentages of calcein-AM positive cells. Values represent a mean of three experiments ±SEM. *p≤0.01. 3D: a bar graph showing the release of lactate dehydrogenase (LDH) by leukemia cell-lines untreated (Ctrl) or treated for 4 h with 20 μg NKexo or with exosomes derived from HEK293 cells (HEKexo). Cytolytic activity was measured by a colorimetric LDH release assay. Bar graph of LDH release levels was calculated as the fold change in OD490 values. Values represent a mean of three experiments ±SEM. *p≤0.05; **p≤0.01. 3E: a graph and a bar graph showing content of NKexo is various fractions of NKexo collected by density gradient centrifugation (upper graph), and LDH release by Jurkat cells following incubation thereof with these fractions. NKexo content of the fractions was determined by nanoparticle tracking analysis (NTA). Cytotoxic activity of each fraction was assessed with LDH release assay;

FIGS. 4A-4B show labeled NKexo uptake by Jurkat cells and immortalized B cells. 4A: confocal microscopy images of Jurkat cells incubated with 5 μg PKH26-labeled NKexo (red; left), or PBS (middle) and immortalized B cells incubated with 5 μg PKH26-labeled NKexo (right). Incubation with labeled NKexo was for 12 h. Cell nuclei were stained with DAPI (blue). The images in the lower panel are superpositions of the images in the upper and middle panels (merge). 4B: a bar graph showing the mean fluorescent intensity (MFI) of PKH26-labeled cells as determined by flow cytometry. Values represent a mean of three experiments ±SEM. **p≤0.001;

FIGS. 5A-5C show NKexo-induced apoptosis in treatment-naïve acute myeloid leukemia (AML), chronic myeloid leukemia (CML), chronic lymphocytic leukemia (CLL) and B cells-acute lymphocytic leukemia (ALL) patient-derived peripheral blood mononuclear cells (PBMCs), and hPBMCs of a healthy donor. The cells were harvested and treated with 20 μg of NKexo for 4 h, or not treated (Ctrl). 5A: representative flow cytometry dot plots of the harvested cells. Apoptosis was evaluated using annexin-V/PI staining. Cells in the lower right quadrant indicate annexin V-positive, early apoptotic cells. The cells in the upper right quadrant indicate annexin-positive/PI-positive, late apoptotic cells or necrosis. Cells in the lower left quadrant indicate annexin-negative/PI-negative, viable cells. 5B: a bar graph showing percentages of apoptotic cells (annexin-V positive) in each sample. 5C: a bar graph showing cytolytic activity assessed in the cells by LDH release assay. Values represent a mean of three experiments ±SEM. *p≤0.05, **p≤0.01;

FIGS. 6A-6B are images (6A) and bar graph (6B) showing NKexo-induced cytotoxicity in PBMCs or bone marrow mononuclear cells (BMNCs) isolated from newly diagnosed CML (n=1) and AML (n=4) patients, respectively. 6A: representative inverted microscope images of colonies of the cells cultured for 14 days in complete methylcellulose semi-solid medium supplemented with 20 μg of NKexo or devoid of NKexo (Ctrl) (4× magnification). 6B: a bargraph showing colony-forming units (CFU) of each patient sample, following the indicated treatment. Colonies were defined as clusters containing >30 cells. Values represent a mean of three experiments ±SEM. *p≤0.01, **p≤0.005. AML, acute myeloid leukemia; and

FIGS. 7A-7C show the in vivo anti-leukemia effect of NKexo. NSG mice were intravenously inoculated with 5×10⁵ HL-60 cells. Following engraftment, xenograft mice were treated with two doses of NKexo: a low dose of 1×10¹⁰ particles/mouse or a high dose of 1×10¹¹ particles/mouse. The doses were administered intravenously in a single dose or in three doses on 3 consecutive days, forming four treatment groups: Single-Low, Triple-Low, Single-High, Triple-High. Control xenograft mice were treated with PBS only. 7A: dot plots of flow cytometry analysis of hCD45+ leukemia blasts in peripheral blood of NSG mice, 7 days after NKexo administration. Shown are representative dot plots of the control and the Triple-High groups. 7B: a bar graph showing percentage of hCD45+ in the different treatment groups. Values represent a mean of three samples ±SEM. *p≤0.01, **p≤0.001. 7C: a Kaplan-Meier curve presenting survival analysis performed using the log-rank statistics to measure the difference between the survival of acute myeloid leukemia xenograft mice treated with Triple-High NKexo (green; n=8) or PBS (black; n=8).

### DETAILED DESCRIPTION

Leukemia is a group of blood cancers that usually begin in the bone marrow and result in high numbers of abnormal blood cells. These blood cells, which are not fully developed, are called blasts or leukemia cells. The symptoms associated with lack of normal blood cells include bleeding and bruising, bone pain, fatigue, fever and an increased risk of infections. Diagnosis is typically made by blood tests and/or bone marrow biopsy.

The exact cause of leukemia is unknown. A combination of genetic factors and environmental factors are believed to play a role. Risk factors include smoking, ionizing radiation, petrochemicals (such as benzene), prior chemotherapy, and Down syndrome. Leukemias and lymphomas both belong to a broader group of tumors that affect the blood, bone marrow, and lymphoid system, known as tumors of the hematopoietic and lymphoid tissues.

There are several types of leukemia, divided based, first, on whether the leukemia is acute (fast growing, aggressive) or chronic (slower growing), and second, whether it starts in myeloid cells (i.e., myelogenous leukemia) or lymphoid cells (i.e., Lymphocytic leukemia). Leukemia classifications are used to break the disease down into the following main types:
(i) Acute lymphocytic leukemia (ALL), also termed acute lymphoblastic leukemia. It is the most common kind of leukemia. It usually occurs in young children but can also occur in adults. ALL starts in the lymphoid cells of the bone marrow. It often spreads quickly to the blood in other parts of the body, such as central nervous system (brain and spinal cord), liver, lymph nodes, spleen, testicles in male patients.
(ii) Acute myelogenous leukemia (AML). The most common kind of aggressive leukemia in adults. It can also affect children. This type of leukemia starts in the myeloid cells of the bone marrow and can spread quickly into the blood. From there, AML can spread to the central nervous system, liver, lymph nodes, spleen, and testicles in male patients. A myeloid cell is a type of blood cell originating in the bone marrow that, when matured into an adult blood cell, will take on a specific role as a basophil, eosinophil, erythrocyte, macrophage, monocyte, neutrophil, or platelet.

AML is also sometimes called: acute granulocytic leukemia, acute myelocytic leukemia, acute myelogenous leukemia, acute myeloid leukemia or acute non-lymphocytic leukemia.
(iii) Chronic lymphocytic leukemia (CLL). The most common type of slow-growing leukemia. It usually affects older adults and accounts for about one-third of all leukemias. CLL starts in the lymphoid (white blood) cells of the bone marrow and progresses slowly. A person with CLL may feel fine for several years before experiencing symptoms or seeking treatment. But it can eventually enter the blood and spread to other parts of the body. CLL is almost completely curable.
(iv) Chronic myelogenous leukemia (CML), also termed chronic myeloid leukemia. It usually affects adults (about 15% of leukemias in adults are CML). This type of leukemia starts in the myeloid cells of the bone marrow and grows slowly, so symptoms may not start for months or years. CML can eventually spread to the blood and other parts of the body.
(v) Chronic myelomonocytic leukemia (CMML). CMML starts in the myeloid cells of the bone marrow and invades the blood. It affects mainly older adults. People with CMML usually have enlarged spleen, shortage of some types of blood cells, too many monocytes. CMML can lead to AML.
(vi) Myeloproliferative neoplasms (MPNs). These neoplasms are a group of blood disorders, wherein the bone marrow produces too many white blood cells, red blood cells or platelets. The type of MPN determines symptoms, treatment options and outlook. There are six types, characterized by which blood cells are abnormal: (1) chronic eosinophilic leukemia: too many eosinophils (white blood cells that fight allergens); (2) chronic myelogenous leukemia: too many white blood cells; (3) chronic neutrophilic leukemia: too many neutrophils (white blood cells that fights infection); (4) essential thrombocythemia: too many platelets; (5) polycythemia vera: too many red blood cells; and (6) primary myelofibrosis, or chronic idiopathic myelofibrosis: abnormal blood cells build up in the bone marrow.

Leukemia is the most common cancer in children and teens, accounting for almost 1 out of 3 cancers. Most childhood leukemias are acute lymphocytic leukemia (ALL). Most of the remaining cases are acute myeloid leukemia (AML). Chronic leukemias are rare in children.

Different types of leukemia have different treatment options and outlooks. Treatment may involve some combination of chemotherapy, radiation therapy, targeted therapy, and bone marrow transplant, in addition to supportive care and palliative care as needed. The success of treatment depends on the type of leukemia and the age of the person.

Chemotherapy is the major form of treatment for leukemia. Depending on the type of leukemia a patient has, he/she may receive a single drug or a combination of drugs. Chemotherapy drugs kill fast-growing cells throughout the body, including both cancer cells and normal, healthy cells. The damage to normal, healthy cells can cause side effects. Chemotherapy is typically given in cycles. Each cycle is made up of a certain number of days of treatment, followed by a certain number of days of rest. Chemotherapy may be orally administered (e.g., when drugs are formulated as pills), or be injected directly into a vein.

Targeted drug treatments focus on specific abnormalities present within cancer cells. By blocking these abnormalities, targeted drug treatments can cause cancer cells to die.

Radiation therapy uses X-rays or other high-energy beams to damage leukemia cells and stop their growth. Radiation may be applied in one specific area of the body where there is a collection of leukemia cells, or over the whole body. Radiation therapy may be used to prepare for a bone marrow transplant.

A bone marrow transplant, also called stem cell transplant, helps reestablish healthy stem cells by replacing unhealthy bone marrow with leukemia-free stem cells that will regenerate healthy bone marrow. Prior to a bone marrow transplant, very high doses of chemotherapy or radiation therapy are applied to destroy the leukemia-producing bone marrow. Then, an infusion of blood-forming stem cells to rebuild the bone marrow is administered. The stem cells may be autologous or from a donor.

Immunotherapy uses one's own immune system to fight cancer. The body's disease-fighting immune system may not attack cancer because the cancer cells produce proteins that “hide” them from the immune system cells. Immunotherapy works by interfering with that process, for example, by engineering the immune cells to fight leukemia. A specialized treatment called chimeric antigen receptor (CAR)-T cell therapy takes one's own germ-fighting T cells, engineers them to fight cancer and infuses them back into the patient's body. Not all types of leukemia are suitable for CAR-T cell therapy.

Exosomes are a class of cell-derived extracellular vesicles of endosomal origin and are typically 30-150 nm in diameter—the smallest type of extracellular vesicle. Enveloped by a lipid bilayer, exosomes are released into the extracellular environment containing a complex cargo of contents derived from the original cell, including proteins, lipids, mRNA, miRNA and DNA.

Multivesicular bodies are unique organelles in the endocytic pathway that function as intermediates between early and late endosomes. This specialized subset of endosomes contains membrane-bound intraluminal vesicles. Intraluminal vesicles are essentially the precursors of exosomes, and form by budding into the lumen of the multivesicular body. Most intraluminal vesicles fuse with lysosomes for subsequent degradation, while others are released into the extracellular space. The intraluminal vesicles that are secreted into the extracellular space become exosomes. This release occurs when the multivesicular body fuses with the plasma membrane.

Exosomes are of general interest for their role in cell biology, and for their potential therapeutic and diagnostic applications. It was originally thought that exosomes were simply cellular waste products, however their function is now known to extend beyond waste removal.

One of the main mechanisms by which exosomes are thought to exert their effects is via the transfer of exosome-associated RNA to recipient cells, where they influence protein machinery. MicroRNAs and long noncoding RNAs are shuttled by exosomes and alter gene expression, while proteins (e.g., heat shock proteins, cytoskeletal proteins, adhesion molecules, membrane transporter and fusion proteins) can directly affect target cells. Like other microvesicles, the function of exosomes likely depends on the cargo they carry, which is dependent on the cell type in which they were produced.

While exosomes are essential for normal physiological conditions, they also act to potentiate cellular stress and damage under disease states. Exosomes have been implicated in a diverse range of conditions including neurodegenerative diseases, cancer, liver disease and heart failure.

In cancer, exosomes have multiple roles in metastatic spread, drug resistance and angiogenesis. Specifically, exosomes can alter the extracellular matrix to create space for migrating tumor cells. Several studies also indicate that exosomes can increase the migration, invasion and secretion of cancer cells by influencing genes involved with tumor suppression and extracellular matrix degradation.

Exosomes are being pursued for use in an array of potential therapeutic applications, such as therapeutically targeted or engineered drug delivery systems. The membranes of these naturally occurring delivery means are tolerated, have low immunogenicity and a high resilience in extracellular fluid making. Furthermore, exosomes bear surface molecules that allow them to be targeted to recipient cells, where they deliver their payload. Exosomes may cross the blood-brain barrier, at least under certain conditions and may be used to deliver an array of therapies including small molecules, RNA therapies, proteins, viral gene therapy and CRISPR gene-editing.

The purification of exosomes is a key challenge in the development of exosome-based therapy. Exosomes must be differentiated from other distinct populations of extracellular vesicles, such as microvesicles (which shed from the plasma membrane) and apoptotic bodies. Although ultracentrifugation is regarded as the gold standard for exosome isolation, it has many disadvantages and alternative methods for exosome isolation are currently being sought.

The content of exosomes represents the cellular makeup of their cell of origin. It has been envisioned by the present inventors that if exosomes derived from NK cells (NK-derived exosomes herein designated “NKexo”) maintain the anti-leukemia capacity of their donor cells, they can serve as a less immunogenic anti-leukemia product with no proliferation capacity that may circumvent the limitations of current cellular therapies. It is shown in embodiments disclosed herein that NKexo exert a potent, selective anti-leukemia effect in vitro, ex vivo and in vivo in several leukemia models.

Furthermore, it has been demonstrated by the present inventors that NKexo eliminated leukemia cells isolated from patients with acute and chronic leukemia and inhibited hematopoietic colony growth. While leukemia cells were targeted and severely affected by NKexo, healthy B-cells remained unaffected, indicating a selective effect. This selectivity was further confirmed by demonstrating that NKexo were specifically taken up by leukemic cells but not by healthy B-cells. In vivo data presented in Example 5 herein support the in vitro and ex vivo findings disclosed in Examples 2-4 herein and demonstrate improved human-CD45⁺ leukemia blast counts and overall survival in NKexo treated humanized acute myeloid leukemia (HL-60) xenograft mice.

In one aspect, the present disclosure relates to a method for treating leukemia, comprising administering to a subject in need thereof an effective amount of NKexo, thereby treating leukemia in the subject.

In some embodiments, the NKexo are obtained from the leukemia patient being treated, namely they are autologous. In accordance with these embodiments, prior to the anti-cancer treatment, NK cells-derived exosomes are collected from the patient using one or more clinical procedures known in the art. For example, a blood sample is taken from the patient and subjected to differential centrifugation including ultracentrifugation (centrifugation at very high speeds). Differential centrifugation attempts to selectively sediment different components. Autologous purified exosomes must be modified or activated in order to make them potent against the leukemic cells of the patient. Engineering techniques such as those use in forming CAR-T cells may be applied to the exosomes in order to increase their killing capacity.

In some embodiments, the NKexo are non-autologous or homologous (allogenic) and obtained e.g., from a compatible donor.

In some embodiments, the NKexo are obtained from an NK cell-line which can be a commercially available NK cell-line.

The dose and regiment of NKexo therapy depend on the type of leukemia, the patient's age and physical condition, and the stage of the disease.

The therapeutically effective amount of exosomes is determined by their total exosomal protein content. For example, one course of treatment may require at least 1 μg of exosomal protein, for example, 1, 1.2, 1.5, 1.6, 1.7, 1.8, 1.9, 2, 2.3, 2.5, 2.6, 2.8, 3, 3.2, 3.4, 3.5, or 4 μg protein exosomal content. The dose of NKexo may be in the range of from about 45×10⁶ to about 80×10⁶ exosomes per kg body weight. For example, a total of about 60×10⁶ exosomes per kg body weight are needed for one course of remission induction in AML patients.

The treatment protocol may comprise one or two courses, each comprising administering a given dose of NKexo once, every 2 or 3 days, or continuously for a week.

Unlike cells, exosomes are not affected by irradiation, therefore their killing capacity is fully preserved. This obvious advantage in the production process would translate to at least one order magnitude less NK cells needed to produce the exosome product compared to a cell product (e.g., CAR-T cells).

Approximately 10⁸ NK cells are needed to produce about 0.75 μg protein of exosomal content.

Any and all types of leukemia may be treated by a disclosed method, including but not limited to, acute lymphocytic leukemia (ALL), chronic lymphocytic leukemia (CLL), chronic myeloid leukemia (CML) and acute myeloid leukemia (AML).

In some embodiments, the disease treatable by a disclosed method is AML. AML patients whose leukemia cells have certain genetic mutations are assigned a specific risk status. With 50% mortality, there is yet an unmet need for an immediate, short term and highly effective therapy, particularly for newly diagnosed patients.

AML treatment consists of three main phases:
(i) Induction therapy;
(ii) consolidation therapy; and
(iii) maintenance.

The first phase of AML treatment is induction therapy. The goal of induction is to destroy as many cancer cells as possible in order to achieve (induce) a complete remission. Typically, initial therapy requires a hospital stay of 4 to 6 weeks.

Almost 50 years have passed since the combination of adriamycin and cytosar was introduced as the first effective strategy to induce remission in patients with AML. This regimen is still considered standard of care (SOC). However, being highly toxic, this combination can only be given to young patients with no significant comorbidities.

Another common induction regimen for AML is the so called the “7+3” regimen, which includes cytarabine and an anthracycline drug, such as daunorubicin or idarubicin, whereby cytarabine is most often given by continuous intravenous (IV) infusion over 7 days, and the anthracycline drug is given by an IV infusion in a single dose for 3 days during the first week of treatment. Other drugs may be added or substituted for these “7+3” drugs for higher-risk patients and targeted therapies, for example: midostaurin (Rydapt®) for FLT3-mutated AML; gemtuzumab ozogamicin (Mylotarg™) for CD33-positive AML; daunorubicin and cytarabine (Vyxeos®) for the treatment of newly diagnosed therapy-related AML or AML with myelodysplasia-related changes in adults; and quizartinib (Vanflyta®) in combination with standard cytarabine and anthracycline induction. Some drugs are given by mouth (orally). Other drugs are inserted directly into the patient's blood stream through a central line, a port or a PICC. Currently, targeted drugs are added to specific subgroups of patients based on molecular features and hypomethylating agents are often given to older patients.

A complete remission is achieved when no more than 5% of cells in the bone marrow are blast cells, blood cell counts are back to normal and/or all signs and symptoms of AML are gone. If the initial treatment does not induce a remission, induction therapy is repeated, either with the same drugs or with a new chemotherapy regimen.

Most patients develop dangerously low blood cell counts and may become very ill, needing supportive (palliative) care with IV antibiotics and frequent blood transfusions during this time.

Even when a complete remission is achieved, some leukemia cells that cannot be seen with a microscope may still remain in the bone marrow. This is referred to as minimal residual disease (MRD), or measurable residual disease. Patients who achieve remission after initial treatment but have MRD are at increased risk of disease relapse. It is important to test for MRD after achieving remission for identifying patients who may benefit from further treatment with intensified therapies, such as allogeneic stem cell transplantation.

“Consolidation therapy,” also termed “post-remission therapy,” is the treatment given after cancer is in remission following induction therapy. The goal of consolidation therapy is to lower the number of residual leukemia cells in the body or eliminate them entirely to prevent recurrence. Without additional therapy, the leukemia is likely to relapse within weeks or months.

Treatment choices for consolidation therapy include additional intensive chemotherapy, allogeneic stem cell transplantation and/or quizartinib in combination with cytarabine. Patients with favorable risk factors are often given intensive chemotherapy with high dose cytarabine and other drugs for their consolidation therapy. Patients with high-risk AML, based on their prognostic factors, receive more aggressive therapy, such as allogeneic stem cell transplantation.

Not all patients can tolerate intensive therapies or even want them. Patients whose comorbidities and performance status make them poor candidates for intensive chemotherapy may benefit from lower-intensity therapies, which may relieve symptoms, improve quality of life and potentially extend survival.

The third phase of treatment is termed “maintenance.” The main objective of maintenance therapy is to deliver a less toxic therapy to prevent relapse after intensive chemotherapy. Maintenance therapy is often an extended course of treatment and depends on the subtype, consolidation treatment and risk of relapse. For example, for some adult patients, azacitidine (Onureg®) or quizartinib (Vanflyta®) may be prescribed as maintenance therapy.

Unfortunately, induction regimens fail in almost 50% of patients. This is why outcomes of AML is still dismal and the survival rate has been only marginally raised during the past 4 decades. Currently, no effective immunologic induction therapy for AML is available or known. Chimeric antigen receptor (CAR) treatment fails to meet the clinical need to produce effective CAR/NK in a very short time, as time is a detrimental factor for AML patients that require immediate and robust induction therapy. Yet, even if this time gap could be overcome, the use of allogeneic source of immune cells would be more effective because autologous NK may fail to recognize the AML cell as non-self and irradicate it. The present inventors have shown in an animal model that exosomes which were kept frozen and then defrosted and administered to the animal enabled effective treatment of AML.

In some embodiments, a disclosed method is applied as an immunologic induction therapy aimed at obtaining immediate remission of newly diagnosed AML patients. In accordance with these embodiments, high amounts of exosomes are administered to the subject either by continuously administering exosomes, for example by continuous drip for at least 3 days, e.g., for 3, 4, 5, 6, or 7 days, or by subsequent (e.g., once daily) administrations of predetermined exosome dosages, once every 2 or three days for a period of 7 days.

The term “high amounts of exosomes”, as used herein refers to a sufficient amount of exosome that yield 1-4 μg of exosomal protein. For example, between 1 to 1.5 μg protein of exosomal content is needed to treat a 35 kg child patient and that between 2 to 3 μg protein exosomal content to treat a 70 kg adult patient.

The exosomal treatment is intended to replace the adriamycin-based chemotherapy. In some embodiments, NKexo are given to an AML patient in a continuous drip, first as an “add on” to SOC, and subsequently as a substitute foradriamycin in the induction phase of AML treatment.

This exosomal-based induction therapy is advantageous over adriamycin treatment. The patients will not be exposed to highly toxic chemotherapy in the induction phase and will not have to endure the short term and long-term side effects of the treatment such as heart failure, infections and the like.

In some embodiments, the exosomes are non-autologous. The non-autologous exosomes may be obtained from an NK cell-line such as commercially available NK cell-lines (e.g., NK92MI). Alternatively, or additionally, the exosomes may be derived from donor NK cells.

The exosomes used may be freshly harvested from NK cells and immediately used. Alternatively, or additionally, the exosomes may be preserved frozen and unfrozen immediately before administration thereof to the patient.

The exosome treatment may be combined with any of the known anti-cancer therapies, including chemotherapy, immunotherapy, bone marrow transplantation, irradiation and the like.

The terms “therapy”, “treatment”, “treating”, “treat” as used herein are interchangeable and refer to: (a) inhibiting leukemia in a subject, i.e., arresting its development; (b) relieving, alleviating or ameliorating leukemia, i.e., causing regression of the cancerous disease; and (c) curing the cancerous disease.

Thus, the terms “treatment”, “therapy” and the like include, but are not limited to, changes in the recipient's status. The changes can be either subjective or objective and can relate to features such as symptoms or signs of leukemia. For example, if the patient notes improvement in overall feeing or decreased pain, then successful treatment has occurred. Similarly, if the clinician notes objective changes, then treatment has also been successful. Alternatively, the clinician may note a decrease in blast counts or other abnormalities upon examination of the patient. This would also represent an improvement or a successful treatment. Preventing the deterioration of a recipient's status is also included in the term. Therapeutic benefit includes any of a number of subjective or objective factors indicating a desirable response of the condition being treated by NKexo.

The term “therapeutically effective amount” as used herein, means the amount or dose of a compound, e.g., NKexo protein, that when administered to a subject according to a method defined herein, is sufficient to effect the intended anti-cancer treatment. The therapeutically effective amount may sometimes be the lowest dose level that yields therapeutic benefit to patients, on average, or to a given percentage of patients. The ‘therapeutically effective amount’ can vary depending on the vitality or potency of the NKexo, the type of leukemia and its severity, the age, weight, etc., of the subject to be treated.

It has been previously shown (Cochran et al. Front Cell Dev Biol., 9:6958639, 2021) that NKexo derived from the NK3.3 cell-line are cytotoxic against malignant hematopoietic cell-lines (K562 and Jurkat). Embodiments described herein verify these findings and, furthermore, provide a more comprehensive analysis of the time- and dose-dependent anti-leukemic activity of NKexo by presenting in vitro as well as ex vivo data from a wider variety of leukemia cell-lines and patient-derived samples. Moreover, the present inventors were the first to provide insight into the anti-leukemic activity of NKexo in vivo, and the first to evaluate the anti-tumoral effect of NKexo on the survival of AML mice model. Several studies have reported the anti-tumoral effect of NKexo in vivo against melanoma-, breast cancer-, glioblastoma-, and neuroblastoma-xenografts. The anti-tumoral activities of NKexo in these studies were presented as a correlation to their effects on tumor growth, viability and apoptosis. However, the effect of NKexo on the survival of the subjected xenograft models was not disclosed.

The terms “comprise”, “comprising”, “includes”, “including”, “having” and their conjugates mean “including but not limited to”.

The term “consisting of” means “including and limited to”.

As used herein, the singular form “a”, “an” and “the” include plural references unless the context clearly dictates otherwise. For example, the term “a compound” or “at least one compound” may include a plurality of compounds, including mixtures thereof.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Various embodiments and aspects as delineated hereinabove and as claimed in the claims section below find experimental support in the following examples.

### EXAMPLES

Reference is now made to the following examples, which together with the above descriptions illustrate some embodiments in a non-limiting fashion.

### Materials and Methods

### (i) Cell Culture

NK92MI, HCC1395BL (B-cells), K562 and UoC cell-lines were obtained from ATCC. HL-60 cells were provided by Dr. Vitaly Kliminsky, FMRC, Rabin Medical Center, Israel. Jurkat cells were provided by Dr. Muayad Zahalka, FMRC. KG-1a cells were provided by Prof. Tsvee Lapidot, Weizmann Institute of Science, Israel. NK92MI cells were cultured in MEM with 12.5% exosome-depleted fetal bovine serum (FBS), 12.5% exosome-depleted donor horse serum, 0.2 mM myo-inositol, 2% exosome-depleted OTC human AB-serum, 0.1 mM 2-mercaptoethanol and 0.02 mM folic acid. HL-60, KG-1a, K562 and B-cells were cultured in IMDM (Sigma-Aldrich) with 10%/20% FBS. Jurkat and UoC cells were cultured in RPMI containing 10% FBS. All media were supplemented with 2 mM 1-glutamine, penicillin (100 U/mL) and streptomycin (100 μg/mL). Unless otherwise stated, all media and supplements were purchased from Sartorious.

### (ii) Patient Samples

Mononuclear cells from bone marrow (BMNCs) or peripheral blood (PBMCs) were obtained from newly diagnosed, untreated acute lymphocytic leukemia (ALL), chronic lymphocytic leukemia (CLL), chronic myeloid leukemia (CML) and acute myeloid leukemia (AML) patients at the Davidoff Cancer Center, Rabin Medical Center, Israel after Informed consent was obtained. Only samples from patients with circulating blast counts exceeding 90% were used.

Healthy donor PBMCs were obtained from members of the Experimental Hematology Lab, FMRC, Rabin Medical Center, Israel. BMNCs or PBMCs were separated by lymphoprep-density gradient (Axis-shield). BMNCs and PBMCs were then shortly cultured in IMDM with 20% FBS and RPMI with 20% FBS, respectively, or cryopreserved in freezing medium consisting of IMDM, 40% FBS and 10% DMSO (Sigma-Aldrich).

(iii) Exosome Isolation

NK92MI cells were cultured in exosome-depleted medium for 48 hours. The conditioned medium was serially centrifuged at 1,500×g/3 min, 2,000×g/15 min and 3,000×g/20 min at 4° C. The supernatant was filtered (0.22 μm) and centrifuged in an ultracentrifuge at 100,000×g/70 min. The pellet was resuspended in 200-400 μl PBS and used immediately or stored at −80° C. Exosome protein content quantity was determined by adding 0.5% NP-40 (Sigma-Aldrich) and using BCA protein assay kit (Thermo Scientific). Nonylphenyl polyethylene glycol (NP-40) is a non-ionic surfactant useful for the isolation and purification of functional membrane protein complexes. The bicinchoninic acid (BCA) assay is utilized to measure the total protein concentration of a sample. In this assay, copper (II) [Cu²⁺] is reduced to copper (1) [Cu¹⁺] in the presence of proteins/peptides in alkaline solutions. BCA can chelate (bind to) Cu¹⁺, creating a purple color with a maximum absorbance at 562 nm. As the protein concentration increases, more of the BCA-Cu¹⁺ complex will form, and the absorbance will increase. This relationship is linear. The colorimetric detection is relative and is compared against a protein standard of known concentration.

### (iv) Nanoparticle Tracking Analysis

Vesicles concentration and size were measured by Nanoparticle Tracking Analysis (NTA) using NanoSight NS300 (Malvern Instruments). For each sample, 3 to 5 60-second videos were recorded and analyzed in the batch processing mode. NTA is a method to visualize and measure nanoparticles in suspension in the range from 10-1000 nm based on the analysis of Brownian motion. Objects with two dimensions smaller than 100 nm are termed nanoparticles or ultrafine particles. The size is the translational diffusion diameter of a sphere, also termed “hydrodynamic diameter”. NTA determines the Brownian motion by analysis of a video sequence. Particles in the sample are visualized by illumination with a laser beam. The scattered light of the particles is recorded with a light sensitive CCD or CMOS camera. Using a special algorithm, particles are detected and their path registered. The size of each individually tracked particle is calculated, thus simultaneously allowing determination of their size distribution and concentration.

### (v) Transmission Electron Microscopy (TEM)

Exosome suspension (10 μl) was fixed in 2% paraformaldehyde. Samples were then adsorbed on formvar/carbon-coated EM grids for 20 min, stained with 2% aqueous uranyl acetate for 5 min and washed with deuterium-depleted water. Samples were examined using JEM 1400 plus transmission electron microscope (Jeol).

Transmission electron microscopes (TEM) use a particle beam of electrons to visualize specimens and generate a highly magnified image. TEMs can magnify objects up to 2 million times. The basic principle of the TEM is that a photographic image is recorded from the electron flux after it has passed through a thin sample of the specimen under study, in which the electrons can be transmitted or diffracted. Transmitted electrons provide bright field images, whereas scattered electrons generate dark-field images revealing structure with higher contrast. TEM provides information about materials at very high spatial resolution, including morphology, size distribution, crystal structure, strain, defects, chemical information down to atomic level and so on.

The grids used in TEM microscopes to place specimens are made of copper with a mesh size of about 3 mm and a layer of plastic called a formvar and a thin film of carbon. The formvar coated TEM grid stabilized with evaporated carbon film is suitable for specimen support, especially for ultra-thin sections. Since the contrast in the electron microscope depends primarily on the differences in the electron density of the organic molecules in the cell, the efficiency of a stain is determined by the atomic weight of the stain attached to the biological structures. Consequently, the most widely used contrast-enhancing stains in electron microscopy are the heavy metals uranium and lead. Uranyl acetate staining enhances the contrast by interaction with lipids and proteins.

### (vi) Exosome Uptake Assay

Exosomes (50-70 μg) were mixed for 4 min with 4 μl PKH26 red fluorescent membrane linker-dye (Sigma-Aldrich) suspended in 0.75 ml diluent, followed by addition of 1% BSA (2 ml) to bind excess dye. PKH26 red fluorescent dye stably binds to the lipid region of cell membrane and emit red fluorescence (Ex/Em=551/567 nm). Samples were then transferred to 100 kDa Amicon® tubes (Merck Millipore) and centrifuged at 3200 g/5-10 min. Thereafter, samples were washed 3×5 ml PBS and transferred to a 7 kDa Zeba™ Spin Desalting column (Thermo Scientific) to remove free dye. For control, PKH26488 dye was mixed with PBS. PKH26-labeled exosomes were examined by confocal microscopy (Leica Microsystems) and FACS analysis.

After confirming successful labeling, cells were incubated with the PKH26-labeled exosomes in 1% FBS medium for 3-4 h. Subsequently, cells were washed 3 times with PBS, seeded on poly-L-lysine-coated cover-slides (Sigma-Aldrich) and covered with FluoroShield™ mounting medium containing DAPI (Abcam) for confocal microscopy analysis. This mounting medium is an aqueous anti-fade mounting medium designed to preserve fluorescence when imaging tissues and cell samples. It is fortified with 4′,6-diamidino-2-phenylindole (DAPI) as a counterstain for DNA.

(vii) Cytotoxicity Induction, Analysis Thereof by FACS and LDH Assay

Cells were cultured in medium containing 1% FBS with NKexo (up to 20 μg) in 96-well plates for 4 h prior to analysis. Apoptosis was assessed using annexin V/PI staining (Abcam) according to the manufacturer's instructions. Analysis was performed on a Gallios flow cytometer and data were processed by Kaluza software (Beckman Coulter). Annexin V-CF Blue PI Apoptosis Staining/Detection Kit contains annexin V labeled with CF Blue, which allows the identification and quantitation of apoptotic cells on a single-cell basis by flow cytometry. Simultaneous staining of cells with Annexin V-CF Blue (blue fluorescence) and the non-vital dye propidium iodide (PI) (orange fluorescence) allows the discrimination of intact cells (Annexin V-CF Blue negative, PI Staining Solution negative), early apoptotic (Annexin V-CF Blue positive, PI Staining Solution negative) and late apoptotic or necrotic cells (Annexin V-CF Blue positive, PI Staining Solution positive).

LDH release was assessed using the CytoTox 96® Non-Radioactive Cytotoxicity Assay (Promeg) according to the manufacturer's protocol. The LDH assay, also known as LDH release assay, is a cell death/cytotoxicity assay used to assess the level of plasma membrane damage in a cell population. Lactate dehydrogenase (LDH) is a stable enzyme, present in all cell types, which is rapidly released into the cell culture medium upon damage of the plasma membrane. LDH is the most widely used marker used to run a cytotoxicity assay.

Released LDH in culture supernatants is measured with a 30-minute coupled enzymatic assay that results in the conversion of a tetrazolium salt (INT) into a red formazan product. The amount of color formed is proportional to the number of lysed cells. Visible wavelength absorbance data are collected using a standard 96-well plate reader. The CytoTox 96® Non-Radioactive Cytotoxicity Assay was used to measure membrane integrity following cell-mediated cytotoxicity by exosomes. This sensitive assay can reveal early, low-level damage to cell membranes that is often missed with other methodologies.

(viii) Calcein-AM Assay

Cells (1×10⁶) were centrifuged at 160 g/3 min and resuspended in 1 ml pre-warmed PBS containing 1 μM calcein-AM (Biolegend). Following 30 min incubation at room temperature, calcein AM-labeled cells were centrifuged at 160 g/3 min. Pellets were washed 2×5 ml PBS to remove excess calcein dye and transferred to prewarmed medium containing 1% FBS. Fluorescent images were captured using a Nikon Eclipse Ti2 inverted microscope. Calcein AM-positive cells were counted using image-J software using at least three different images of each replicate.

The calcein AM cell viability assay is an endpoint analysis method for cell viability. Calcein-acetoxymethyl ester (Calcein-AM) is a fluorogenic, membrane-permeant fluorescent probe that indicates cellular health. When the acetoxymethyl ester is intact, this probe is nonfluorescent. Cleaving the AM ester by nonspecific esterases present within the healthy, live cells generates the green fluorescence dye calcein, which is highly negatively charged and is well-retained in cell cytoplasm. The probe excites and emits at 488 nm/520 nm respectively. The signal of calcein-AM is proportional to cell vitality, as esterase activity decreases in cells with poor vitality.

### (ix) Western Blotting

Western blotting is an electrophoretic technique used to move proteins from a gel onto a solid support, such as a nitrocellulose or PVDF membrane. The membrane can be used for immunological or biochemical analyses or demonstration of protein-protein or protein-ligand interactions.

Cells were lysed in RIPA buffer for 30 min on ice. After centrifugation (15000 g/5 min), supernatants were separated on Mini-PROTEAN® precast gels (Bio-Rad) and transferred to nitrocellulose membranes (Bio-Rad). Mini-PROTEAN® precast gels are 7.2 cm×8.6 cm gels designed for performing polyacrylamide gel electrophoresis.

After transfer, blots were prepared for immunodetection by placing the membrane into Tris-buffered saline with Tween 20 (TTBS) containing blocking agent (for example, 3% BSA, 5% nonfat dry milk, 1% casein, or 1% gelatin) and incubated (1 hour at room temperature or overnight at 4° C.). The primary antibodies were diluted in blocking solution and incubated with the blot overnight at 4° C. The blot was washed with TTBS. Then, the secondary antibodies were diluted into TTBS as specified by the manufacturer, and the blot was incubated in the secondary solution at room temperature with agitation for 1 hr. Lastly, the blot was washed with TTBS five times, 5 min each at room temperature with agitation.

Signals were detected using the Odyssey Imaging System (LI-COR). Primary antibodies: mouse anti-human CD63, mouse anti-human CD81, rabbit anti-human Alix, rabbit anti-human Caspase-3 (Cell Signaling). Secondary antibodies: IRDye 800CW/680RD (LI-COR). A loading control is an antibody specific for a ubiquitously and constitutively expressed protein and used to normalize protein levels in Western blot. Loading control antibodies assess equal loading of samples across a gel. Constitutively expressed proteins include, for example, β-actin, β-tubulin, or GAPDH. The loading control antibody used in embodiments described herein was mouse anti-α-tubulin (Sigma-Aldrich).

### (x) Colony-Forming Assay

PBMCs or BMNCs (1×10⁴) were incubated at 37° C. in complete methylcellulose medium with recombinant cytokines (MethoCult™ H4330; Stemcell Technologies) supplemented with PBS or 20 μg NKexo. The MethoCult™ H4330 medium is suitable for the growth and enumeration of hematopoietic progenitor cells in colony-forming unit (CFU) assays of human bone marrow, and peripheral blood samples. Colonies were counted after 14 days with an inverted microscope. Colonies were defined as clusters containing >30 cells.

(xi) OptiPrep™ density gradient purification

OptiPrep™ is a density gradient medium for the isolation and purification of particles such as DNA or RNA viruses, nuclei, mitochondria, endosomes or exosomes, macromolecules, as well as a wide range of cell types. OptiPrep™ is a flexible, versatile, non-ionic iodixanol-based medium with a density of 1.320±0.001 g/ml. The high density of OptiPrep™ facilitates the fractionation of cells by flotation from a dense load zone through either a continuous or discontinuous gradient.

A discontinuous iodixanol gradient was prepared by diluting a stock solution of OptiPrep™ (60% w/v) (Stemcell Technologies) with 0.25 M sucrose/10 mM Tris, pH 7.5 to generate 40%, 20%, 10% and 5% w/v iodixanol solutions. Iodixanol gradient was generated by sequentially layering 3 ml of each of these solutions, followed by 2.5 ml of 5% iodixanol solution in 1489 mm Ultra-Clear™ Beckman Coulter centrifuge tubes. A 500 ml volume of conditioned medium particles was overlaid on the gradient and centrifuged using a SW 40 Ti rotor for 16 h at 100,000 g (k-factor: 277.5) at 4° C. The gradient allowed the particles to be separated based on their density and size, with heavier or denser particles settling towards the bottom of the tube and lighter or less dense particles remaining towards the top.

Fractions of 1 ml were collected from the top of the gradient and introduced to Vivaspin® 20 centrifugal concentrator tubes (Merck) to reduce sample volume to 80 μl. Samples were then analyzed for particle concentration.

(xii) In Vivo Animal Study

Animal protocols were approved by the Israeli Ministry of Health Animal Experimentation Committee. To generate a humanized xenograft AML model, nonobese diabetic/severe combined immunodeficiency IL2Rg-null (NSG) mice (6-8 weeks old) were treated with 20 mg/kg busulfan (Sigma-Aldrich) by intraperitoneal administration 24 h before injection of leukemic cells. HL-60 cells were washed twice in PBS, cleared of aggregates and debris using a 0.2 mm cell filter and suspended in PBS at a final concentration of 5×10⁵ cells/200 μl PBS. Xenograft tumors were generated by inoculating these HL-60 cells intravenously. Engraftment was determined by monitoring levels of human CD45⁺ (hCD45⁺) cells in mice peripheral blood via FACS analysis. Once the transplants were engrafted and leukemia was initiated, NKexo were intravenously administered at different doses and regimens.

CD45 is an antigen found on the surface of all nucleated hematopoietic cells. A given cell is said to be “CD45 positive” (CD45⁺) if an isoform of the CD45 antigen is present on its surface. For hCD45⁺ cell count, peripheral blood was obtained via retroorbital blood draws. Fifty microliter of blood was collected in heparin-coated collection tube. Samples were then exposed to red blood cell lysis buffer (BioLegend) to lyse erythrocytes. Harvested PBMCs were then subjected to FACS analysis.

(xiii) Statistical Analysis

Unless differently indicated, Student's t test was used. Statistical significance was assumed at p≤0.01 or p≤0.005. Each experiment was repeated at least 3 times.

### Example 1

### NK-Derived Exosomes Isolation and Characterization

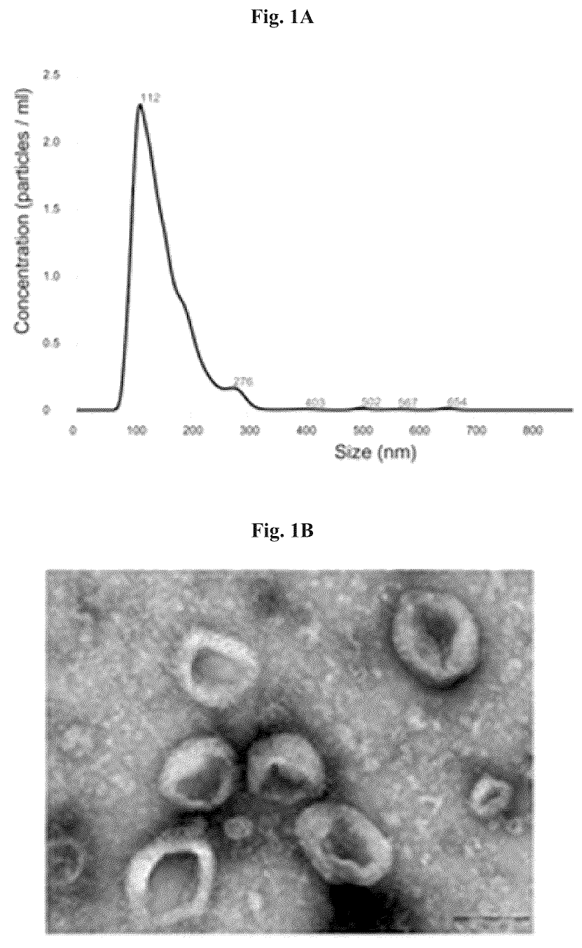

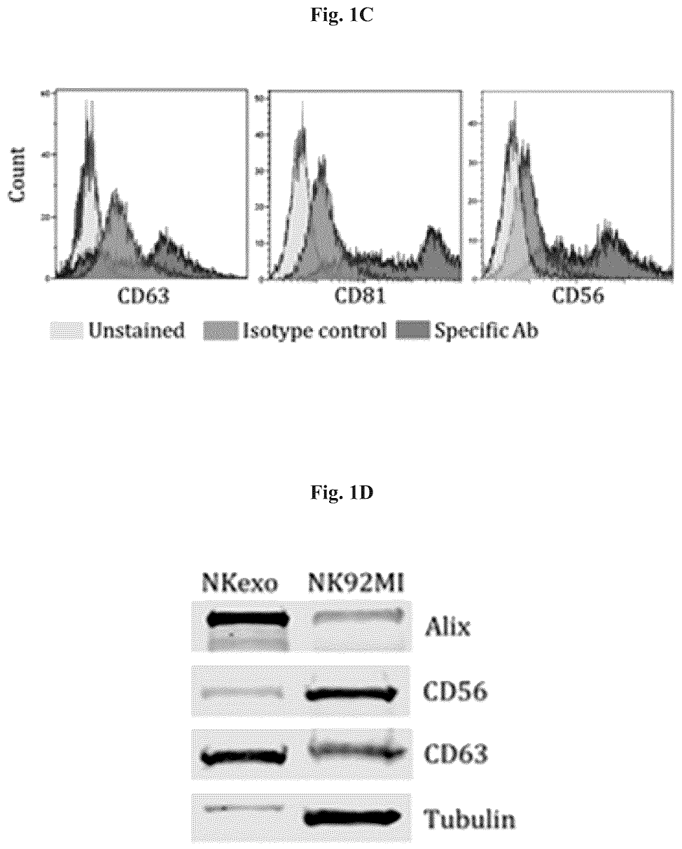

Extracellular vesicles (EVs) from the supernatant of NK92MI cells were purified by differential centrifugation and ultracentrifugation (UCF) and the secreted EVs were characterized in terms of concentration, size, morphology and membrane markers. Using nanoparticle tracking analysis (NTA), it was observed that NK92MI cells secrete large amounts of 70-200 nm sized particles ranging between ˜1 and 3.8×10¹¹ particles/ml (with an average of 2.4×10¹¹±1.02×10¹¹ particles/ml) (FIG. 1A).

Transmission electron microscopy (TEM) further confirmed the presence of 100-200 nm membrane-bound particles featuring a typical exosomal shape as shown in FIG. 1B. FACS and Western blotting established the presence of the exosomal proteins CD63, CD81 and Alix, and the NK-derived protein CD56, as shown in FIGS. 1C and 1D, respectively. The purified exosome fraction was in fact enriched with the exosomal proteins Alix and CD63 (FIG. 1D). These data confirm the successful isolation of EVs presenting exosomal morphological and biochemical features from NK92MI cell medium. These EVs will be termed NKexo throughout this manuscript.

### Example 2

### Induction of Selective Cell Death of Leukemia Cell-Lines by NK-Derived Exosomes

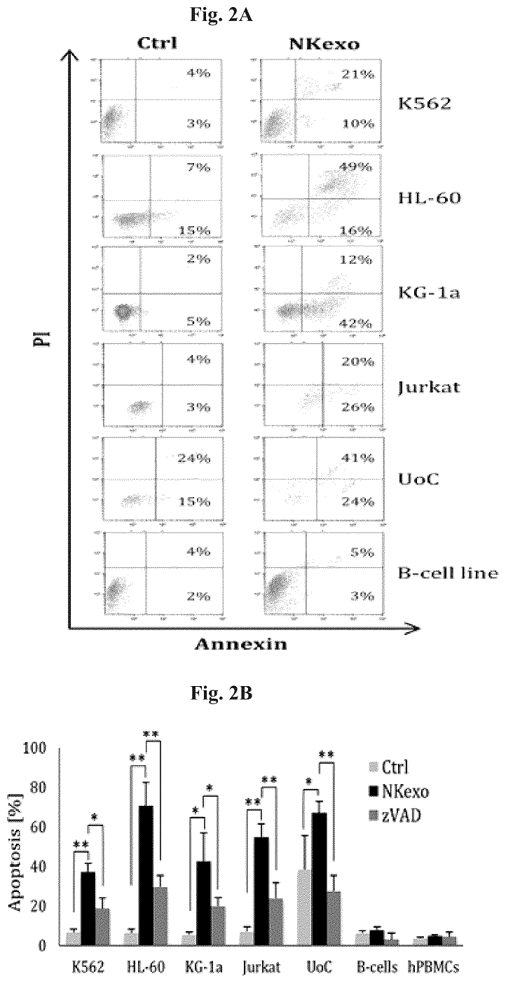

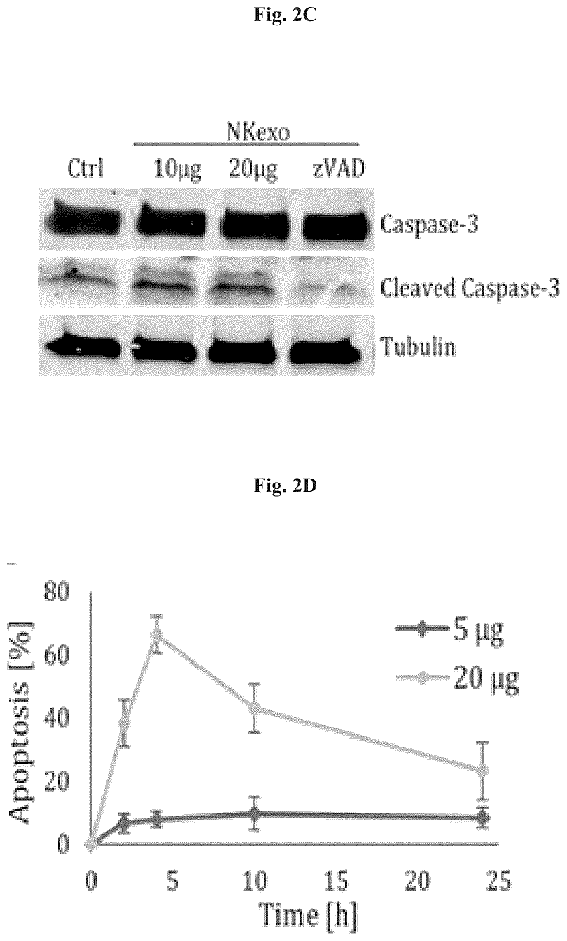

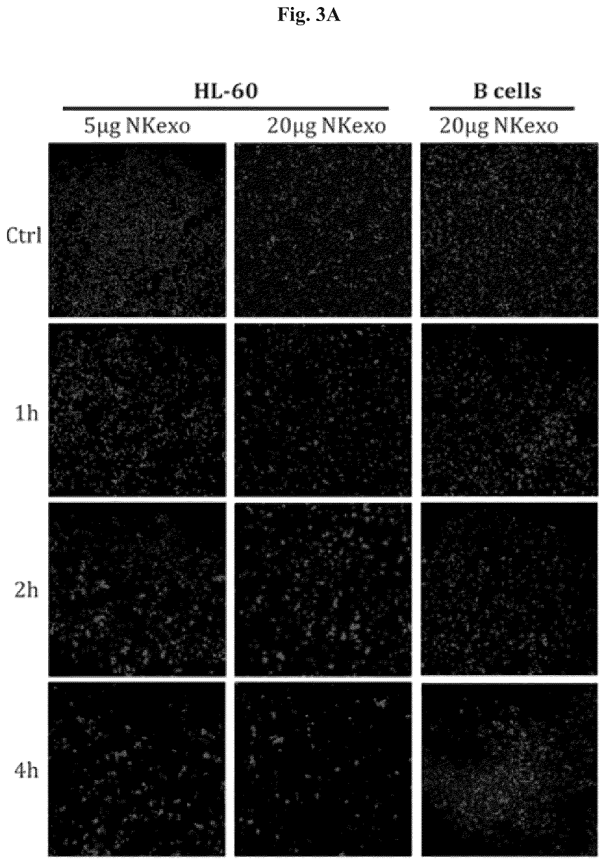

Cytotoxicity in various leukemic cell lines was induced by NKexo and assessed by various means as described in Materials and Method. NKexo (20 μg) increased apoptosis rates by up to 64.37±11.7% across all tested cells (K562 (CML); HL-60 and KG1a (AML); Jurkat and UoC (ALL)), as shown in FIGS. 2A and 2B. In contrast, immortalized B-cells and healthy-donor PBMCs (hPBMCs) remained unaffected by NKexo, suggesting a selective cytotoxic effect specific to leukemia cells (FIGS. 2A-2B).

Caspases are a family of intracellular cysteine proteases critical to several cellular functions, including apoptosis and inflammation. Mammalian caspases are numbered 1-13 and are classified either as initiators or effectors of downstream functions. Caspases are present as inactive pro-enzymes that are activated by proteolytic cleavage. Caspases-8, -9 and -3 play a central role in programmed cell death and are essential regulators and mediators of the apoptotic process, orchestrating the orderly disassembly or dismantling of cells. Caspase-8 and Caspase-9 are initiator caspases whereas Caspase-3 is an effector caspase, i.e., it is a key executioner, responsible for initiating and executing the ordered breakdown of cellular components. Upon activation (inter alia by the action of Caspases-8 and -9), Caspase-3 undergoes proteolytic cleavage, giving rise to cleaved Caspase-3, the active form critical for mediating apoptosis and propagating the apoptotic signal by targeting downstream substrates like poly ADP ribose polymerase (PARP) and other cellular proteins.

Caspase-3 antibodies are often applied to detect both the uncleaved and cleaved forms of the enzyme, making them strong indicators of cell death induction. Herein, rabbit anti-human Caspase-3 antibodies were used to assess the cytotoxic potency and effectiveness of NKexo. As shown in FIG. 2C, increased levels of cleaved Caspase-3 reinforced the involvement of the caspase-pathway in the NKexo-induced apoptotic response.

Pan-caspase inhibitors act on one or more of the known caspases and are pursued for their ability to treat diseases such as autoimmune disorders and cancer. Pan-caspase inhibitors can either be peptides, proteins, or small molecule inhibitors. The compound z-VAD-FMK (z-VAD) is a cell-permeable pan-caspase inhibitor. It potently inhibits human Caspase-1 to-10 with the exception of Caspase-2. It also inhibits murine caspases, notably Caspase-1, Caspase-3, and Caspase-11, the ortholog of human Caspase-4 and -5.

Like their cells of origin, NKexo have been shown to contain cytotoxic molecules, such as FasL, perforin, granulysin, granzymes-A, —B, and —K, which are responsible for NKexo-mediated activation of several caspases and for the subsequent induction of apoptosis in their target cells. Accordingly, the present inventors detected the presence of these cytotoxic proteins (except granzyme-B, proteomics data not shown) and others in the exosomes isolated from NK-cells.

Co-incubating the leukemic cells with the pan-caspase inhibitor z-VAD partially reversed NKexo-induced apoptosis, as shown in FIG. 2B, stressing the contribution of caspase-dependent apoptosis to the cytotoxic activity of NKexo and that NKexo treatment induced the cleavage of caspases-3.

Exposing HL-60 cells to increasing concentrations of NKexo for 2-24 h revealed an obvious dose-dependent and a partial time-dependent apoptotic effect. In FIG. 2D, two representative doses of NKexo are shown. Interestingly, incubation of HL-60 cells with 20 μg NKexo for 2 and 4 h increased apoptotic death of these cells by 38.2±7.5% and 66.26±5.8% (p≤0.05), respectively, however their apoptosis rates were reduced following 10 and 24 h of NKexo treatment. The decrease in apoptosis is attributed to a decrease in cell number due to potential cytolytic activity of the exosomes.

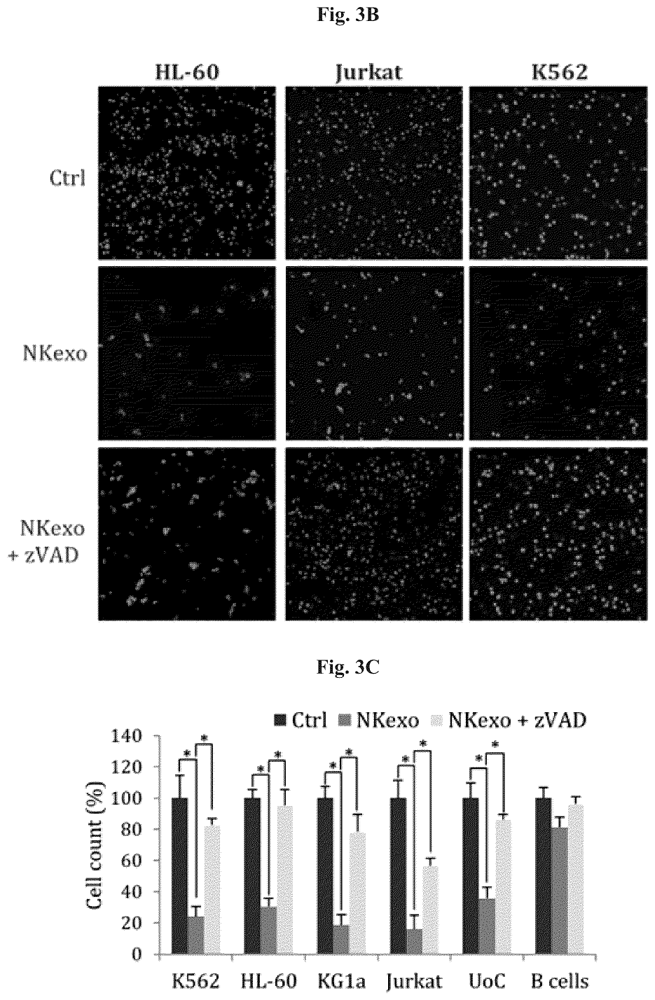

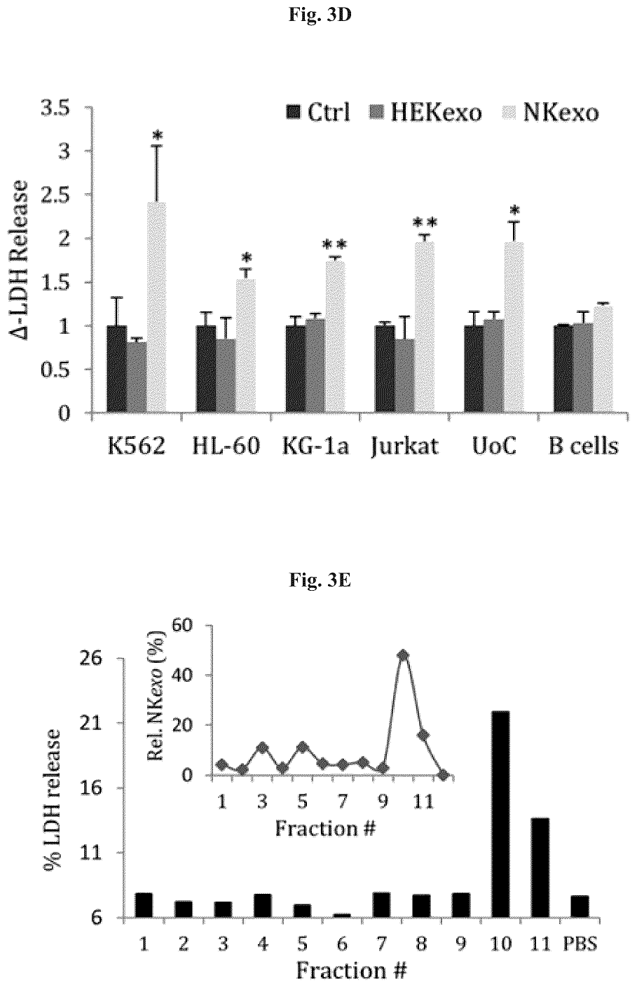

NK-cells are known to possess cytolytic activity via the release of cytotoxic effectors (i.e., perforin). A calcein-AM-based assay was used to determine whether NKexo possess this ability as well. The assay was conducted as described in Materials and Methods herein above, and the results are shown in FIGS. 3A-3C.

HL-60 counts were indeed reduced by 42.1±4% and 69.6±3.3% (p≤0.001) after 4 h of exposure to 5 or 20 μg exosomes (FIG. 3A), respectively. A similar reduction in cell count, ranging between 65% and 84% was seen in all leukemia cell-lines tested (FIGS. 3B, 3C). Exposing immortalized B-cells to the same dose of NKexo did not affect their number, indicating once again the selectivity of NKexo (FIGS. 3A, 3C).

The cytotoxic effect of NKexo was completely attenuated by z-VAD, indicating, once again, caspase-dependency (FIGS. 3B, 3C).

In order to assess the level of plasma membrane damage, the lactate dehydrogenase (LDH) release assay was conducted (as described in Materials and Methods). The enzyme LDH is rapidly released into the cell culture medium upon damage of the plasma membrane. The results are shown in FIG. 3D.

LDH release assay confirmed the cytolytic activity of NKexo towards all leukemia cells tested but not towards immortalized B-cells (FIG. 3D).

HEK293 cells are clonal isolates derived from adenovirus type 5 transformed embryonal human kidney cells expressing the E1A adenovirus gene. Exosomes derived from these cells were evaluated for their cytotoxic activity against various leukemic cells. Notably, equivalent doses of HEK293-derived exosomes did not affect any of the tested leukemia cells (FIG. 3D).

To further prove specificity, NKexo samples were fractionated by OptiPrep™ density gradient as described in Materials and Methods. Then, Jurkat cells were exposed to equal volumes of NKexo fractions collected by the density gradient. NKexo content of the fractions was determined by nanoparticle tracking analysis (NTA), and the relative amounts of NKexo in several fractions are presented in the upper graph of FIG. 3E. The cytotoxic activity of each fraction was assessed with LDH release assay. As shown in the lower bar graph of FIG. 3E, only NKexo-bearing fractions exerted cytotoxic activity against leukemia cells, attributing the cytotoxic effect solely to NKexo and not to other EVs or additional contaminants.

Taken together, it has been shown herein that exosomes derived from NK-cells, but not from HEK293-cells, exert a selective potent anti-leukemic effect that eventually translates into cytolysis of the targeted leukemic cells.

### Example 3

### NKexo Uptake by Leukemia Cells

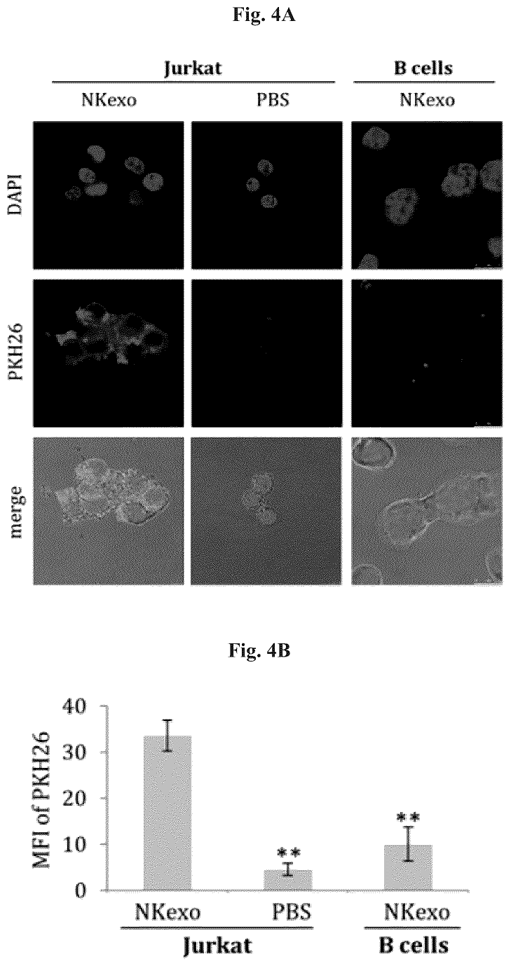

To assess NKexo uptake, Jurkat and immortalized B-cells were incubated with PKH26-labeled NKexo for 3-4 h as described in Materials and Methods. The results are shown in FIGS. 4A-4B.

Labeled-NKexo was found in the perinuclear regions of Jurkat cells but not of the B-cells, indicating selective cellular association and uptake of NKexo (FIG. 4A). Incubating Jurkat cells with the PKH26 dye in PBS alone ruled out contamination with un-bound dye and non-specific transfer to the cells. Additionally, following incubation with PKH26-labeled NKexo, the mean fluorescence intensity of PKH26-positive Jurkat cells peaked at 33.6±5.2, indicating high uptake rates (FIG. 4B). In contrast, immortalized B-cells displayed only slight uptake aptitude with a mean fluorescent intensity (MFI) of 9.9±7.2.

This preferential NKexo-uptake and the differential cytotoxic effect observed reinforce the conception of a selective targeting ability of NKexo towards leukemia cells.

### Example 4

### Induction of Selective Cell Death of Primary Leukemia Cells by NKexo

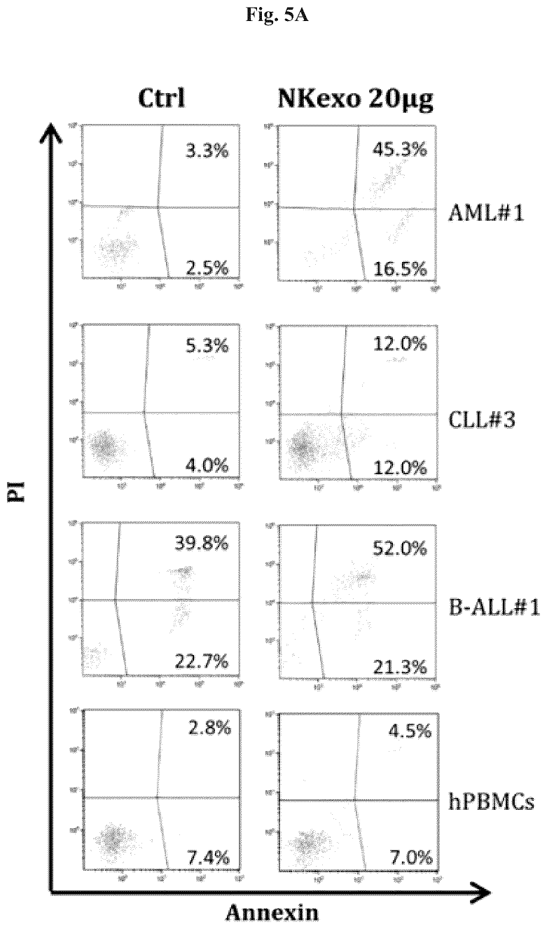

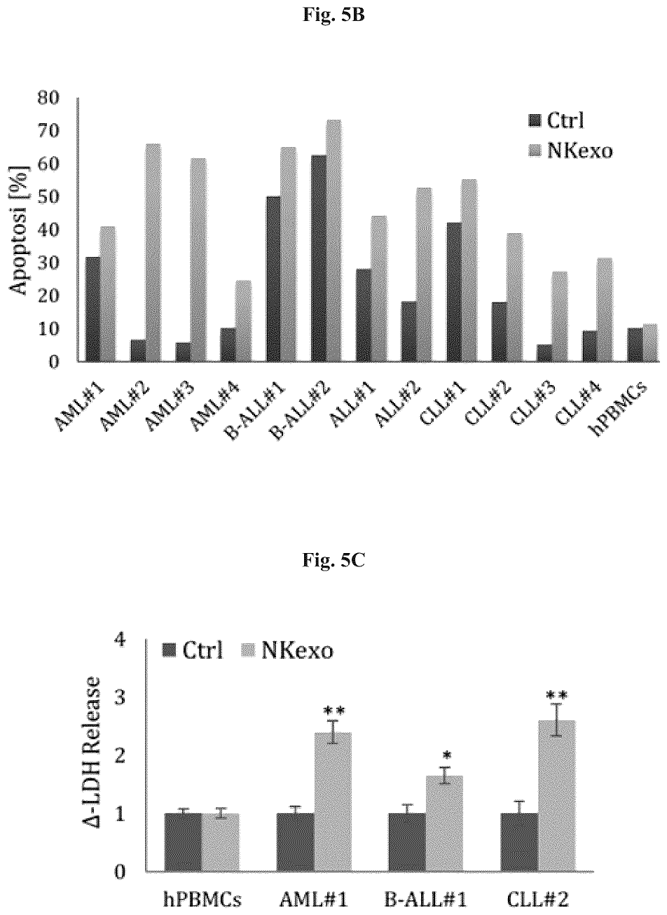

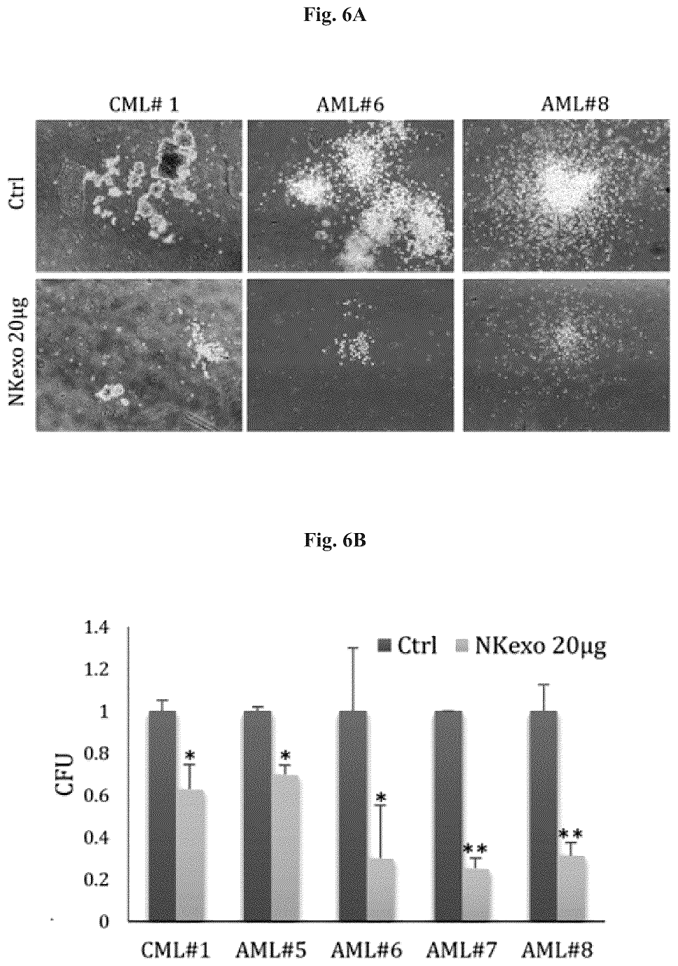

The potency of NKexo to induce apoptosis and inhibit colony formation and growth of patient-derived leukemia cells was assessed as described in Materials and Methods. The results are shown in FIGS. 5A-5C and FIGS. 6A-6B.

When tested against PBMCs derived from newly diagnosed, untreated AML, CLL and B-ALL patient, NKexo (20 μg, 4 h) significantly increased apoptosis rates by 10%-60% as seen in FIGS. 5A and 5B, and resulted in a 2.39±0.19-, 1.65±0.14- and 2.6±0.27-fold increase in LDH release, respectively. This suggests that NKexo trigger cytolysis of primary cells (FIG. 5C). Healthy donor PBMCs showed no considerable increase in apoptosis or LDH release (FIGS. 5A-5C).

In addition, the clonogenic potential of treatment-naïve CML-derived PBMCs (n=1) and AML-derived BMNCs (n=4) was significantly reduced following exposure to 20 μg NKexo for 14 days. The relative colony-formation efficiency of CML cells was reduced by an average of 28±14% (p≤0.005) while the colony formation ability of AML cells was even more compromised and decreased by up to 75±13% (p≤0.005) (FIGS. 6A, 6B). Notably, the colonies that did form in the presence of NKexo appeared smaller and contained less cells.

### Example 5

### In Vivo Assessment of the Anti-Leukemia Effect of NKexo

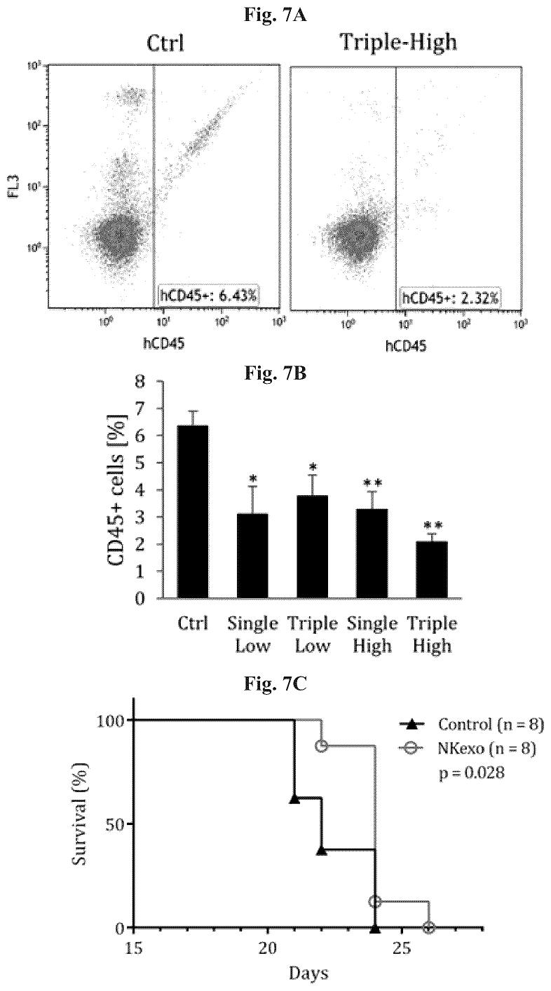

A humanized AML xenograft model was generated by injecting NSG mice (n=40) with HL-60 cells. Following engraftment, mice were randomly divided into four treatment groups and one control group (n=8/group). Treatment group mice were intravenously injected with low (1×10¹⁰ particles/mouse) or high (1×10¹¹ particles/mouse) NKexo doses, administered in a single dose or as three doses given on 3 consecutive days, forming four treatment groups specified as Single-Low, Triple-Low, Single-High, Triple-High. The control group was administered PBS for 3 consecutive days. The results are shown in FIGS. 7A-7C.

After only 7 days of treatment, the number of hCD45⁺ leukemia blasts found in control mice peripheral blood was significantly higher than that found in peripheral blood of the treatment groups, suggesting that NKexo limited leukemia expansion. This was most prominent in the Triple-High treatment group where the number of hCD45⁺ leukemia blasts was 2.1±0.8% as opposed to 6.3±0.5% in the control group (p≤0.001, FIGS. 7A-7B). In addition, after 26 days of monitoring, increased survival of the Triple-High treatment group was evident as shown in the Kaplan-Meier curve (Log-rank: p=0.028; FIG. 7C).

Despite the small cohort of mice, these data support the in vitro and ex vivo findings and demonstrate improved hCD45⁺ leukemia blast counts and overall survival in NKexo treated mice, thus, supporting, for the first time, the assumption that NKexo possess an anti-leukemia effect.

What is claimed is:

1. A method for treating leukemia, comprising administering to a subject in need thereof therapeutically effective amount of natural killer cells (NK)-derived exosomes, thereby treating leukemia in the subject.

2. The method of claim 1, wherein the therapeutically effective amount of NK-derived exosomes accounts for 1 to 4 μg exosomal protein per treatment course.

3. The method of claim 1, wherein the NK-derived exosomes are autologous.

4. The method of claim 1, wherein the NK-derived exosomes are allogenic.

5. The method of claim 4, wherein the NK-derived exosomes are from a living donor or derived from an NK cell-line.

6. The method of claim 1, wherein the NK-derived exosomes are fresh or preserved exosomes.

7. The method of claim 1, wherein the leukemia is selected from the group consisting of acute lymphocytic leukemia (ALL), chronic lymphocytic leukemia (CLL), chronic myeloid leukemia (CML), chronic myelomonocytic leukemia (CMML) and acute myeloid leukemia (AML).

8. A method for induction therapy in a subject afflicted with acute myeloid leukemia (AML), comprising administering therapeutically effective amount of natural killer cells (NK)-derived exosomes to the subject to induce remission.

9. The method of claim 8, wherein the therapeutically effective amount of NK-derived exosomes accounts for 1 to 3 μg exosomal protein per treatment course.

10. The method of claim 8, wherein the NK-derived exosomes are non-autologous.

11. The method of claim 10, wherein the NK-derived exosomes are derived from an NK cell-line.

12. The method of claim 8, wherein the NK-derived exosomes are fresh or preserved exosomes.

13. The method of claim 8, further combined with standard of care anti-cancer therapy.

14. The method of claim 13, wherein the anti-cancer therapy is selected from the group consisting of chemotherapy, immunotherapy, irradiation and bone marrow transplantation.
